(19) United States
(12) Patent Application Publication
(10) Pub. No.: US 2022/0355121 A1
(43) Pub. Date: Nov. 10, 2022

(54) SYSTEM AND METHOD FOR IDENTIFYING A RECIPIENT OF AN IMPLANTABLE SENSORY PROSTHESIS

(71) Applicant: Cochlear Limited, Macquarie University, NSW (AU)

(72) Inventor: Matthew Zygorodimos, Macquarie University, NSW (AU)

(21) Appl. No.: 17/754,084

(22) PCT Filed: Oct. 9, 2020
(86) PCT No.: PCT/IB2020/059528

§ 371 Date: Mar. 23, 2022

Related U.S. Application Data
(60) Provisional application No. 62/914,734, filed on Oct. 14, 2019.

(51) Int. Cl. A61N 1/372; A61N 1/05; G06F 21/35; H04L 9/08

(52) U.S. Cl. CPC A61N 1/37252; A61N 1/0541; A61N 1/37223; G06F 21/35; H04L 9/0866; H04L 2209/88; H04L 2209/80

(57) ABSTRACT

An apparatus includes a housing configured to be implanted in or on a recipient. The apparatus further includes circuitry within the housing, the circuitry including at least one storage device configured to store at least one secret. The circuitry is configured to generate, using the at least one secret, at least one code corresponding to the at least one secret and to transmit at least one stimulation signal to the recipient, the at least one stimulation signal indicative of the at least one code.

### BACKGROUND

### Field

The present application relates generally to implantable medical systems, and more specifically to implantable sensor prostheses configured to communicate information to the recipients of the implantable sensory prostheses.

### Description of the Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In one aspect disclosed herein, an apparatus comprises a housing configured to be implanted in or on a recipient. The apparatus further comprises circuitry within the housing, the circuitry comprising at least one storage device configured to store at least one secret. The circuitry is configured to generate, using the at least one secret, at least one code corresponding to the at least one secret and to transmit at least one stimulation signal to the recipient, the at least one stimulation signal indicative of the at least one code.

In another aspect disclosed herein, an apparatus comprises at least one first communication interface configured to wirelessly communicate with a system comprising at least one implant in or on a recipient. The implant is configured to generate, using at least one secret, at least one code corresponding to the at least one secret and to transmit at least one stimulation signal to the recipient, the at least one stimulation signal indicative of the at least one code. The apparatus further comprises at least one second communication interface configured to receive at least one user input signal from the recipient, the at least one user input signal indicative of the at least one code. In certain embodiments, the at least one first communication interface is further configured to transmit at least one trigger signal to the at least one implant, the at least one trigger signal configured to initiate said generating the at least one code by the at least one implant.

In yet another aspect disclosed herein, a method comprises accessing at least one secret stored on a device implanted in or on a recipient. The method further comprises generating at least one first code corresponding to the at least one secret. The method further comprises transmitting at least one stimulation signal to the recipient, the at least one stimulation signal indicative of the at least one first code.

In yet another aspect disclosed herein, a method comprises transmitting at least one trigger signal to a device implanted in or on a recipient. The device is configured to respond to the at least one trigger signal by: using at least one secret stored on the device to generate at least one code and transmitting at least one stimulation signal indicative of the at least one code to the recipient. The method further comprises receiving at least one first signal indicative of a perceived at least one code perceived by the recipient in response to the at least one stimulation signal. The method further comprises transmitting at least one second signal indicative of the perceived at least one code. The method further comprises receiving at least one comparison signal indicative of whether the at least one code and the perceived at least one code match one another or not. The method further comprises either providing the recipient with access to a restricted functionality in response to the at least one comparison signal being indicative of the at least one code matching the perceived at least one code or not providing the recipient with the access in response to the at least one comparison signal being indicative of the at least one code not matching the perceived at least one code.

### BRIEF DESCRIPTION OF THE DRAWINGS

Embodiments are described herein in conjunction with the accompanying drawings, in which:

FIG. 1 is a perspective view of an example cochlear implant auditory prosthesis implanted in a recipient in accordance with certain embodiments described herein;

FIG. 2A schematically illustrates an example apparatus in accordance with certain embodiments described herein;

FIG. 2B schematically illustrates an example apparatus comprising a cochlear implant auditory prosthesis in accordance with certain embodiments described herein;

FIGS. 3A-3C schematically illustrate example configurations in which the example apparatus can be used in accordance with certain embodiments described herein;

FIG. 4A schematically illustrates an example on-line use configuration in which the example apparatus can be used in accordance with certain embodiments described herein;

FIG. 4B schematically illustrates an example off-line use configuration in which the example apparatus can be used in accordance with certain embodiments described herein;

FIGS. 5A-5B are flow diagrams of example methods for authenticating a recipient's identity in the example on-line use configuration of FIG. 4A in accordance with certain embodiments described herein; and

FIGS. 6A-6B are flow diagrams of example method for authenticating a recipient's identity in the example off-line use configuration of FIG. 4B in accordance with certain embodiments described herein.

### DETAILED DESCRIPTION

Many systems require that users identify themselves prior to providing the users with access to a restricted capability of the system. For example, in response to a request from a user to access a service from the system (e.g., to access a secured internet portal or website; to perform an electronic transaction; to make an online electronic payment) or to access a real-world secured device (e.g., to operate/open a smartphone, electronic tablet, remote control, lock, door), the system can attempt to authenticate the user's claimed identity, to confirm that the user is permitted to receive the service. Authentication is typically performed by requesting information that can only be provided by the individual with the claimed identity. Such information can include, but is not limited to: a password which is assumed to be known only by the claimed individual; a key with is assumed to be held only by the claimed individual; and/or biometric dataset (e.g., fingerprint; retinal pattern) which is assumed to be available only to the claimed individual. The system provides the restricted capability to the user upon one or more correct passwords, keys, and/or biometric data sets.

However, each of these types of information have attributes which can limit its effectiveness as an authentication tool. For example, passwords can be guessed (e.g., by repeated attempts), and this limitation can be mitigated by including minimal lengths of passwords and denial of access after a limited number of guesses. Also, passwords, once known, can be exposed to others and/or used until revoked, and this limitation can be mitigated by forcing password resets after a limited amount of time. For another example, keys can be stolen, transferred between users, and/or duplicated once acquired, and these limitations can be mitigated by requiring the use of new keys after a limited amount of time. For another example, biometric data sets can be either too sensitive (e.g., the individual cannot accurately reproduce the data set) or not sufficiently specific (e.g., other individuals are able to reproduce the data set). While these limitations may be overcome in a general sense, such measures can be at the expense of usability by the individual which can limit the individual's voluntary compliance with recommendations for good security management.

Certain embodiments described herein advantageously utilize the unique relationship between a sensory prosthesis and a recipient of the sensory prosthesis to facilitate authentication of the recipient's identity (e.g., to third parties such as web service providers) without the use of passwords. In particular, the sensory prosthesis serves as a communication pathway that provides information to the recipient in a manner that is difficult if not impossible to be discerned by anyone except the recipient. For example, a code provided to the recipient through the recipient's perception of stimulation signals from the sensory prosthesis is difficult to be guessed by an unauthorized entity but easy to be recalled by the recipient. In addition, from the recipient's point of view, the sensory prosthesis provides the recipient with a password at the time of authentication that only utilizes a limited mental burden to remember (e.g., does not need to be remembered after the time of authentication).

Certain embodiments described herein utilize a secret residing within an implanted sensory prosthesis and to generate, using the secret, a code that is communicated to the recipient through stimulation signals from the implanted sensory prosthesis. In this way, knowledge of the code is an ability uniquely afforded to the recipient alone. Highly sophisticated attacks (e.g., attempts by third parties to discern the code) are possible, but such attacks are very detectable by the recipient as they likely require explantation or electrodes on the recipient's skin. The secret can be known by an authority (e.g., the implanted sensory prosthesis itself; a remote server) to facilitate confirmation whether a code received from a user corresponds to authorized access by the user of the restricted capability.

While various embodiments are described herein by citing smartphones and websites as examples of devices which can be used, the systems and methods described herein are not so limited. Certain embodiments also extend to bank safes, door locks, and any other objects that use some form of security to access, and other forms of mobile personal devices (e.g., mobile phones; smart phones; electronic tablets). Certain embodiments described herein can be used to provide higher security to access any secured or sensitive information or object. For example, certain embodiments can be used to allow users be identified and verified as being among those individuals who are authorized to access information that is secured or that is sensitive.

The teachings detailed herein are applicable, in at least some embodiments, to any type of implantable medical device (e.g., implantable sensory prostheses) configured to communicate information to the recipient of the implantable medical device. For example, the implantable medical device can comprise an auditory prosthesis system utilizing an implantable actuator assembly that generates electrical, optical, and/or vibrational stimulation signals to the recipient that are perceived by the recipient as sounds. Examples of auditory prosthesis systems compatible with certain embodiments described herein include but are not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Embodiments can include any type of medical device that can utilize the teachings detailed herein and/or variations thereof. In some embodiments, the teachings detailed herein and/or variations thereof can be utilized in other types of implantable medical devices beyond auditory prostheses. For example, the concepts described herein can be applied to any of a variety of implantable medical devices comprising an implanted component configured to provide stimulation signals (e.g., electrical, optical and/or vibrational stimulation signals) to the recipient of the implanted component so as to communicate information to the recipient of the implanted component. For example, such implantable medical devices can include one or more of the following: visual prostheses (e.g., retinal implants); brain implants; seizure devices (e.g., devices for monitoring and/or treating epileptic events); sleep apnea devices; functional electrical stimulation devices.

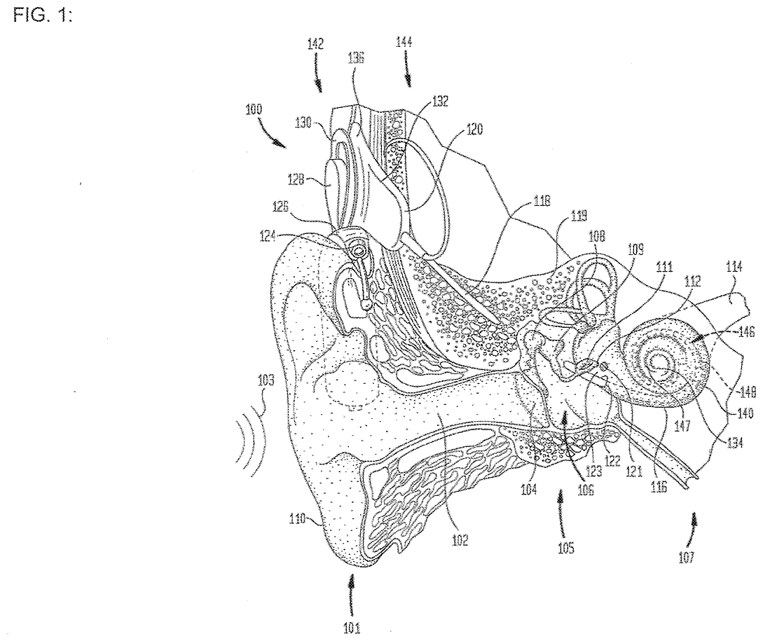

FIG. 1 is a perspective view of an example cochlear implant auditory prosthesis 100 implanted in a recipient in accordance with certain embodiments described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain embodiments described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other embodiments, the sound processing unit 126 has other arrangements, such as by an OTE processing unit (e.g., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

The sound processing unit 126 of certain embodiments includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative embodiments of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted embodiment of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some embodiments, the internal receiver unit 132 and the stimulator unit 120 are hermetically sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some embodiments, the electrode assembly 118 can be implanted at least in the basal region 116, and sometimes further. For example, the electrode assembly 118 can extend towards an apical end of the cochlea 140, referred to as the cochlea apex 134. In certain circumstances, the electrode assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other embodiments, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

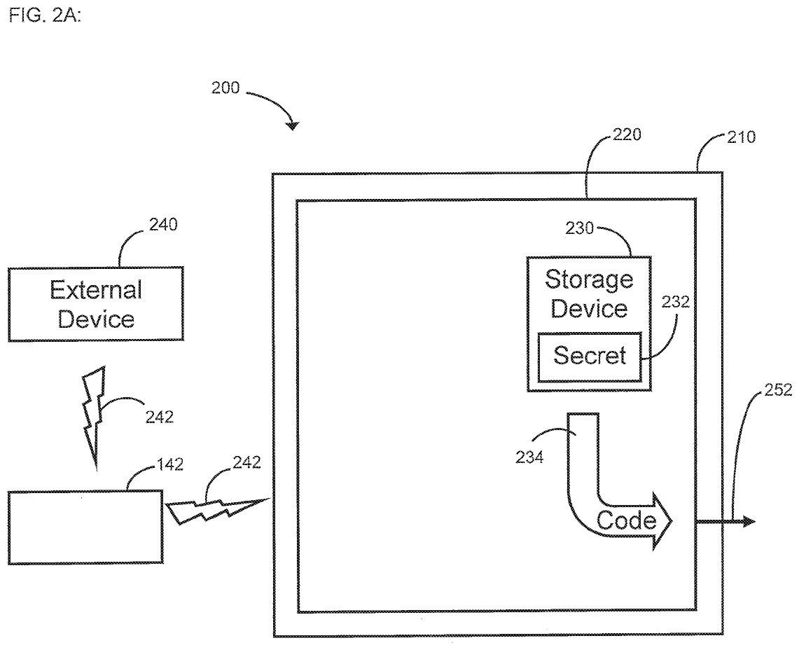

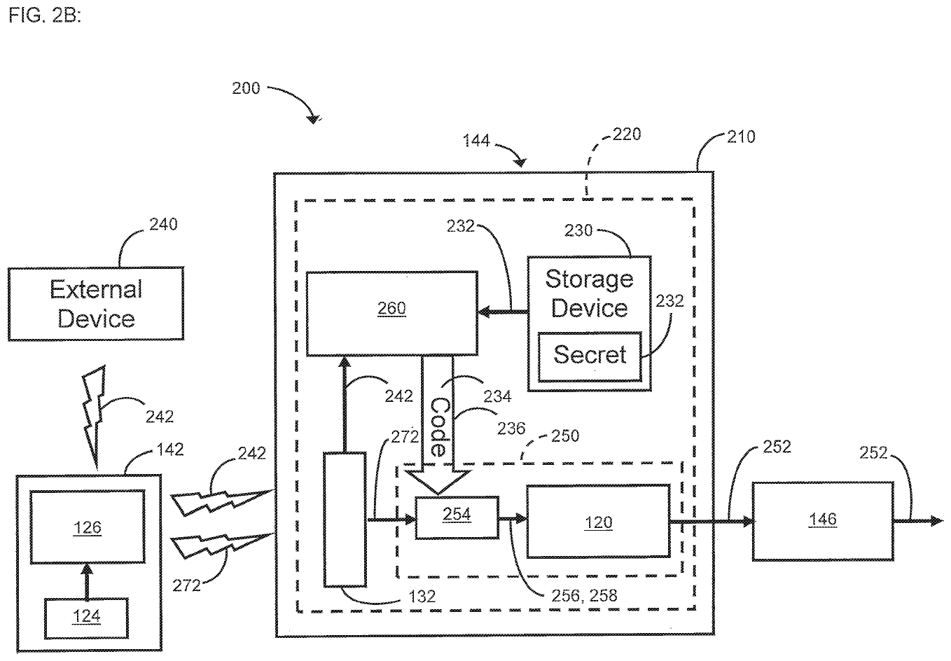

FIG. 2A schematically illustrates an example apparatus 200 (e.g., an implantable sensory prosthesis) in accordance with certain embodiments described herein. The apparatus 200 comprises a housing 210 configured to be implanted in or on a recipient. The apparatus 200 further comprises circuitry 220 within the housing 210, the circuitry 220 comprising at least one storage device 230 configured to store at least one secret 232. The circuitry 220 is configured to generate, using the at least one secret 232, at least one code 234 corresponding to the at least one secret 232 and transmit at least one stimulation signal 252 to the recipient, the at least one stimulation signal 252 indicative of the at least one code 234. In certain embodiments, the circuitry 220 is configured to wirelessly receive at least one trigger signal 242 from an external device 240 and, in response to the received at least one trigger signal 242, to generate the at least one code 234 and to transmit the at least one stimulation signal 252.

In certain embodiments, the apparatus 200 comprises an implantable medical device (e.g., implantable sensory prostheses) configured to communicate information to the recipient of the implantable medical device. For example, the apparatus 200 can comprise an auditory prosthesis utilizing an implantable actuator assembly. FIG. 2B schematically illustrates an example apparatus 200 comprising a cochlear implant auditory prosthesis 100 (see, e.g., FIG. 1) in accordance with certain embodiments described herein. The example apparatus 200 shown in FIG. 2B comprises an internal component 144 and an external component 142, the external component 142 comprising a sound processing unit 126 and an external microphone assembly 124. As schematically illustrated by FIG. 2B, the internal component 144 of the cochlear implant auditory prosthesis 100 comprises the housing 210 and the circuitry 220, and the circuitry 220 comprises the at least one storage device 230, an internal receiver unit 132 in wireless communication with the external component 142, sound processing circuitry 250, and control circuitry 260 in accordance with certain embodiments described herein.

In certain embodiments, the housing 210 comprises a biocompatible material, examples of which include but are not limited to: silicone; polyurethane; polyethylene terephthalate (PET); polyimide; polyether ether ketone (PEEK); titanium; platinum; nitinol; thermoplastic polymer resin; thermoplastic elastomer. The housing 210 of certain embodiments further comprises an inner region containing the circuitry 220, the inner region hermetically sealed from an outer region outside the housing 210. The housing 210 of certain embodiments is configured to be implanted between the recipient's skull and skin tissue (e.g., adhered to or affixed to a surface of the recipient's skull).

In certain embodiments, the at least one storage device 230 comprises non-volatile memory (e.g., flash memory) circuitry in operable communication with the control circuitry 260. As described herein, the at least one storage device 230 is configured to store at least one secret 232 and to provide the at least one secret 232 to the control circuitry 260 (e.g., upon request by the control circuitry 260).

In certain embodiments, the external device 240 comprises a mobile device (e.g., mobile personal device; smart electronic device; smartphone; electronic tablet; remote control) configured to be carried by the recipient and/or kept by the recipient in proximity to the recipient. As schematically illustrated in FIG. 2A, the external device 240 of certain embodiments comprises at least one communication interface configured to wirelessly communicate with an external component 142 of the apparatus 200 which is configured to wirelessly communicate with the circuitry 220. For example, the external device 240 can comprise at least one communication interface (e.g., circuitry configured to perform wireless communications via RF, Bluetooth, WiFi, etc.) in wireless communication with the external component 142 of the auditory prosthesis 100 of FIG. 1, and the external component 142 can be in wireless communication with the circuitry 220 (e.g., via an inductive and/or RF link between at least one external inductive communication coil 130 of the external component 142 and at least one internal inductive communication coil 136 of the internal receiver unit 132). In certain other embodiments, the external device 240 is configured to be in wireless communication with the circuitry 220 directly (e.g., without a separate external component 142 in the communication path between the external device 240 and the circuitry 220).

In certain embodiments, the sound processing circuitry 250 is located within the implantable housing 210 and comprises at least one processor 254 (e.g., microelectronic circuitry; sound processor; digital signal processor) and a stimulator unit 120, as schematically illustrated by FIG. 2B. The at least one processor 254 of certain embodiments comprises at least one integrated circuit configured to receive signals 272 from the external component 142 (e.g., via the internal receiver unit 132) indicative of sounds detected by the microphone 124 and to process the signals 272 (e.g., to apply one or more of digitization, shifting, shaping, amplification, compression, filtering, and/or other signal conditioning to the signals 272). The at least one processor 254 is further configured to transmit the processed signals 256 to the stimulator unit 120. The stimulator unit 120 of certain embodiments is configured to respond to the processed signals 256 from the at least one processor 254 and to generate and transmit the stimulation signals 252 to a portion of the auditory system of the recipient (e.g., the cochlea 140) via the electrodes 148 of the electrode array 146, thereby stimulating the auditory nerve 114. The recipient can perceive the stimulation signals 252 as sounds from the recipient's environment.

While FIG. 2B schematically illustrates the sound processing circuitry 250 and the at least one storage device 230 as separate components, other configurations are also compatible with certain embodiments described herein (e.g., the sound processing circuitry 250 and the at least one storage device 230 being integrated with one another). While FIG. 2B schematically illustrates the at least one processor 254 and the stimulator unit 120 as separate components, other configurations are also compatible with certain embodiments described herein (e.g., the at least one processor 254 and the stimulator unit 120 being integrated with one another).

In certain embodiments, the control circuitry 260 is located within the implantable housing 210 and comprises at least one processor (e.g., microelectronic circuitry; digital signal processor) configured to receive the at least one trigger signal 242 from the external device 240 (e.g., via the external component 142 of the apparatus 200 and the internal receiver unit 132 of the circuitry 220, as schematically illustrated by FIG. 2B). In certain embodiments, the control circuitry 260 is configured to respond to the at least one trigger signal 242 by accessing the at least one secret 232 from the at least one storage device 230 and generating, using the at least one secret 232, the at least one code 234 corresponding to the at least one secret 232. The control circuitry 260 of certain embodiments transmits at least one signal 236 indicative of the at least one code 234 to the sound processing circuitry 250 (e.g., the at least one processor 254). The sound processing circuitry 250 is configured to respond to the at least one signal 236 by transmitting at least one stimulation signal 252 to the recipient, the at least one stimulation signal 252 indicative of the at least one code 234. For example, the at least one processor 254 can be configured to, in response to the at least one signal 236 (e.g., which was generated by the control circuitry 260 in response to the at least one trigger signal 242), generate and transmit at least one signal 258 indicative of the at least one code 234 to the stimulator unit 120. The stimulator unit 120 can be configured to respond to the at least one signal 258 from the at least one processor 254 by generating stimulation signals 252 indicative of the at least one code 234 and transmitting the stimulation signals 252 to a portion of the auditory system of the recipient (e.g., the cochlea 140) via the electrodes 148 of the electrode array 146, thereby stimulating the auditory nerve 114. The recipient can perceive the stimulation signals 252 as sounds (e.g., a voice speaking the at least one code 234) which communicate the at least one code 234 to the recipient. For example, the control circuitry 260 can access audio data (e.g., stored on the at least one storage device 230) corresponding to samples of a voice speaking each of the alphanumeric characters that can possibly be included in the at least one code 234, and the control circuitry 260 can generate the at least one signal 236 by concatenating the appropriate audio data corresponding to voice samples which speak the at least one code 234.

While FIG. 2B schematically illustrates the control circuitry 260 and the at least one storage device 230 as separate components, other configurations are also compatible with certain embodiments described herein (e.g., the control circuitry 260 and the at least one storage device 230 being integrated with one another). While FIG. 2B schematically illustrates the control circuitry 260 and the sound processing circuitry 250 as separate components (e.g., the at least one processor 254 of the sound processing circuitry 250 separate from the at least one processor of the control circuitry 260), other configurations are also compatible with certain embodiments described herein (e.g., the control circuitry 260 and the sound processing circuitry 250 being integrated with one another).

In certain embodiments, the at least one secret 232 is stored within the apparatus 200 prior to implantation of the apparatus 200 (e.g., at the time of fabrication of the apparatus 200). The at least one secret 232 of certain embodiments comprises an alphanumeric string (e.g., 128 bits; 256 bits; 512 bits; serial number or other information indicative of an identity of the apparatus 200) that is assigned to the apparatus 200 and stored within the apparatus 200 (e.g., by the at least one storage device 230). In certain such embodiments, the at least one code 234 is generated (e.g., by the circuitry 200; by the control circuitry 260) using the at least one secret 232 by applying at least one predetermined algorithm to the at least one secret 232 (e.g., inputting the at least one secret 232 into at least one algorithm configured to output the at least one code), the at least one algorithm comprising a series of operations (e.g., one or more modulo operations; one or more truncation operations; one or more concatenation operations; one or more mathematical operations) applied to the at least one secret 232 and the resulting information used as the at least one code 234. In certain embodiments, the at least one algorithm used to generate the at least one code 234 using the at least one secret 232 can be modified periodically (e.g., rotated at regular intervals, at irregular intervals, and/or after a predetermined time period; modified upon request by the recipient and/or by the entity providing the restricted functionality) and/or expired after a predetermined time period. In certain embodiments, the at least one secret 232 comprises a private key and the at least one code comprises a public key.

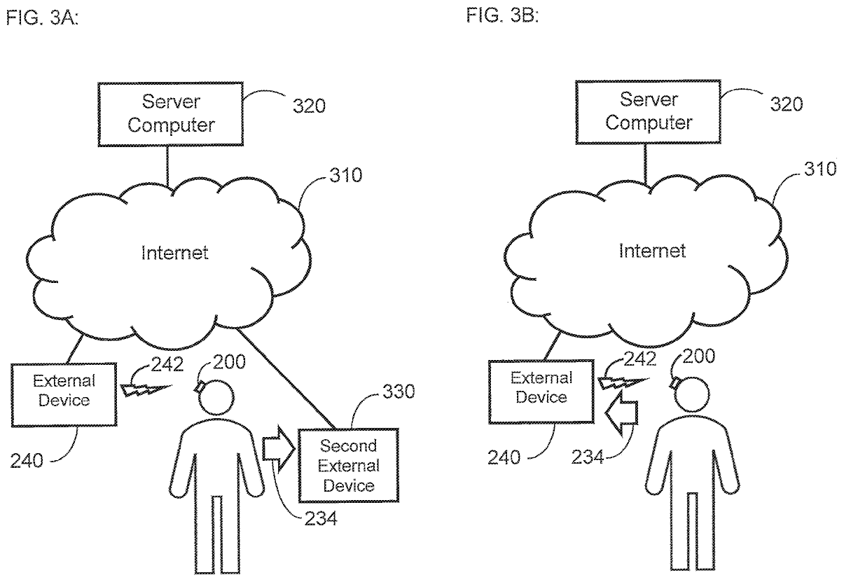

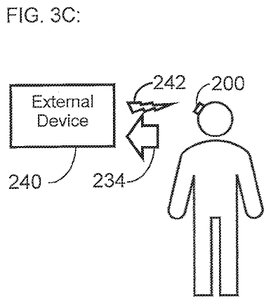

FIGS. 3A-3C schematically illustrate example configurations in which the example apparatus 200 can be used in accordance with certain embodiments described herein. As schematically illustrated by FIGS. 3A-3B, in certain embodiments, at least one server computer 320 is also in operative communication (e.g., wireless; wired) with a network 310 (e.g., the internet). The at least one server computer 320 is configured to provide at least one restricted functionality (e.g., accessing secured information; conducting a secure transaction) which the recipient is allowed to utilize once the recipient's identity is authorized. In certain embodiments, the at least one server computer 320 further comprises a verification server computer (e.g., a computing device configured to participate in the authentication process and in communication with another server computer configured to provide the at least one restricted functionality). The external device 240 in wireless communication with the apparatus 200 is in operative communication (e.g., wireless; wired) with the network 310 such that the external device 240 is configured to communicate with the at least one server computer 320 via the network 310.

In certain embodiments, as schematically illustrated by FIG. 3A, the recipient is using a second external device 330 (e.g., personal computer; laptop computer; notebook computer; electronic tablet) that is in operative communication (e.g., wireless; wired) with the network 310 and that is configured to communicate with the at least one server computer 320. For example, the second external device 330 can be running a web browser program (e.g., Internet Explorer®, Firefox®, Safari®) to access or visit at least one website hosted by the at least one server computer 320 via the network 310. In certain such embodiments, the at least one server computer 320 receives a request from the recipient (e.g., via the second external device 330) to use the second external device 330 to access a restricted functionality hosted by the at least one server computer 320, and the at least one server computer 320 responds by initiating an authentication process to confirm the recipient's identity.

For example, the at least one server computer 320 can respond to the request by transmitting an authentication initiation signal to the external device 240 (e.g., via the network 310) and the external device 240 can respond by generating and transmitting the at least one trigger signal 242 to the apparatus 200. The apparatus 200 can, in response to the at least one trigger signal 242, generate the at least one code 234 corresponding to the at least one secret 232 of the apparatus 200 and can transmit the at least one code 234 to the recipient (e.g., via at least one stimulation signal 252) and to the at least one server computer 320 (e.g., via the external device 240 and the network 310). The recipient can then communicate at least one perceived code to the second external device 330 (e.g., provide at least one user input signal indicative of the at least one perceived code via a keyboard, touchpad, mouse, microphone, or other input communication interface of the second external device 330), and the second external device 330 can communicate the at least one perceived code to the at least one server computer 320 (e.g., via the network 310).

In certain embodiments, the at least one server computer 320 then compares the at least one code 234 received from the apparatus 200 and the at least one perceived code received from the second external device 330 to determine whether the recipient is the individual attempting to access the restricted functionality. For example, if the at least one code 234 and the at least one perceived code match, the at least one server computer 320 can provide access to the restricted functionality. If the at least one code 234 and the at least one perceived code do not match, the at least one server computer 320 can communicate the failed authentication to the second external device 330 and not provide access to the restricted functionality. The second external device 330 can communicate the failed authentication to the recipient (e.g., via a display, speaker, or other output communication interface of the second external device 330). In certain other embodiments, the external device 240 performs the comparison of the at least one code 234 received from the apparatus 200 and the at least one perceived code received from the second external device 330 (e.g., sent to the external device 240 by the at least one server computer 320 via the network 310).

As schematically illustrated by FIG. 3B, in certain embodiments, the second external device 330 is not used, but the external device 240 comprises a mobile device (e.g., mobile phone; smartphone; electronic tablet) that is running an application that accesses or visits at least one website hosted by the at least one server computer 320 via the network 310, and the recipient is using the external device 240 to communicate with the at least one server computer 320. For example, the external device 240 can be running a web browser program (e.g., Internet Explorer®, Firefox®, Safari®) to access or visit at least one website hosted by the at least one server computer 320 via the network 310. In certain such embodiments, the at least one server computer 320 receives a request from the recipient (e.g., via the external device 240) to use the external device 240 to access a restricted functionality hosted by the at least one server computer 320, and the at least one server computer 320 responds by initiating an authentication process to confirm the recipient's identity.

For example, the at least one server computer 320 can respond to the request by transmitting an authentication initiation signal to the external device 240 (e.g., via the network 310) and the external device 240 can respond by generating and transmitting the at least one trigger signal 242 to the apparatus 200. The apparatus 200 can, in response to the at least one trigger signal 242, generate the at least one code 234 corresponding to the at least one secret 232 of the apparatus 200 and can transmit the at least one code 234 to the recipient (e.g., via at least one stimulation signal 252) and to the at least one server computer 320 (e.g., via the external device 240 and the network 310). The recipient can then communicate at least one perceived code to the external device 240 (e.g., provide at least one user input signal indicative of the at least one perceived code via a keyboard, touchpad, mouse, microphone, or other input communication interface of the external device 240), and the external device 240 can communicate the at least one perceived code to the at least one server computer 320 (e.g., via the network 310).

In certain embodiments, the at least one server computer 320 then compares the at least one code 234 received from the apparatus 200 and the at least one perceived code received from the external device 240 to determine whether the recipient is the individual attempting to access the restricted functionality. For example, if the at least one code 234 and the at least one perceived code match, the at least one server computer 320 can provide access to the restricted functionality. If the at least one code 234 and the at least one perceived code do not match, the at least one server computer 320 can communicate the failed authentication to the external device 240 and not provide access to the restricted functionality. The external device 240 can communicate the failed authentication to the recipient (e.g., via a display, speaker, or other output communication interface of the external device 240). In certain other embodiments, the external device 240 performs the comparison of the at least one code 234 received from the apparatus 200 and the at least one perceived code received from the recipient.

As schematically illustrated by FIG. 3C, in certain embodiments, the external device 240 comprises a mobile device (e.g., mobile phone; smartphone; electronic tablet) that is running a local application (e.g., without needing communications to the network 310 and/or the at least one server computer 320 of FIGS. 3A-3B. For example, the external device 240 can be running a local application which authenticates the recipient's identity prior to allowing the recipient to access a restricted capability hosted hosted by the external device 240 (e.g., access to interact with an application that controls operation of the external device 240). In certain such embodiments, the external device 240 receives a request from the recipient to access the restricted functionality hosted by the external device 240, and the external device 240 responds by initiating an authentication process to confirm the recipient's identity.

For example, the external device 240 can respond to the request from the recipient by generating and transmitting the at least one trigger signal 242 to the apparatus 200. The apparatus 200 can, in response to the at least one trigger signal 242, generate the at least one code 234 corresponding to the at least one secret 232 of the apparatus 200 and can transmit the at least one code 234 to the recipient (e.g., via at least one stimulation signal 252) and to the external device 240. The recipient can then communicate at least one perceived code to the external device 240 (e.g., provide at least one user input signal indicative of the at least one perceived code via a keyboard, touchpad, mouse, microphone, or other input communication interface of the external device 240). In certain embodiments, the external device 240 then compares the at least one code 234 received from the apparatus 200 and the at least one perceived code received from the recipient to determine whether the recipient is the individual attempting to access the restricted functionality. For example, if the at least one code 234 and the at least one perceived code match, the external device 240 can provide access to the restricted functionality. If the at least one code 234 and the at least one perceived code do not match, the external device 240 can communicate the failed authentication to the recipient (e.g., via a display, speaker, or other output communication interface of the external device 240) and not provide access to the restricted functionality.

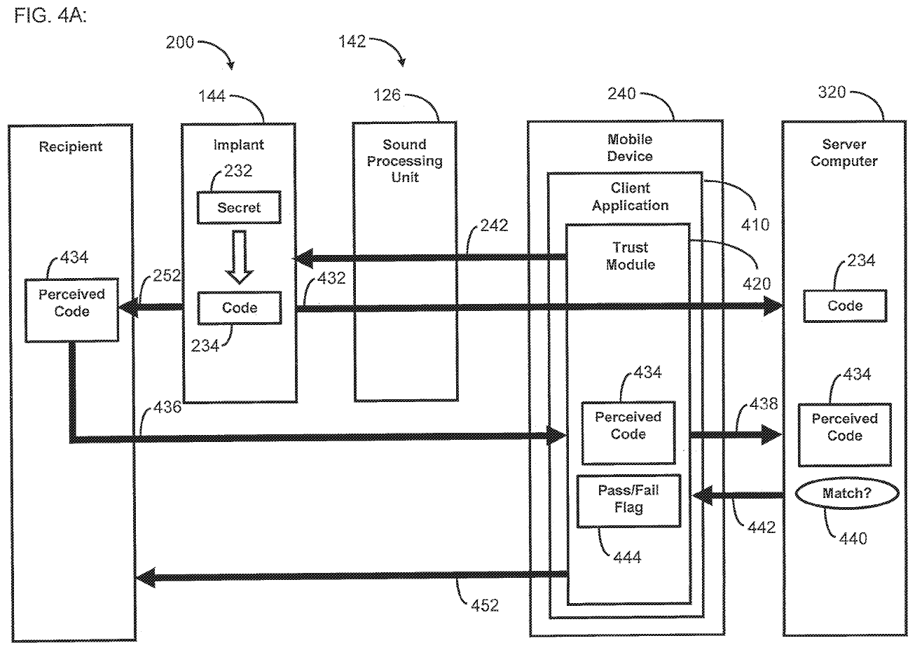

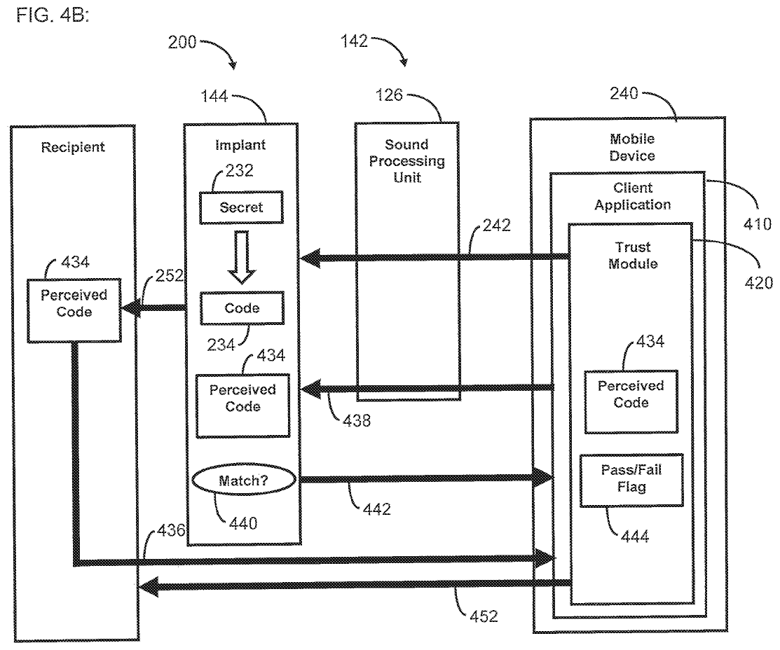

FIG. 4A schematically illustrates an example on-line use configuration in which the example apparatus 200 can be used in accordance with certain embodiments described herein. FIG. 4B schematically illustrates an example off-line use configuration in which the example apparatus 200 can be used in accordance with certain embodiments described herein. In FIGS. 4A-4B, the apparatus 200 comprises an implanted internal component 144 of a sensory prosthesis system (e.g., a cochlear implant auditory prosthesis 100) that comprises an external component 142 in communication with the implanted internal component 144, and the external device 240 comprises a mobile device running a client application 410 with a trust module 420 configured to facilitate the authentication of the recipient's identity in accordance with certain embodiments described herein. In FIGS. 4A-4B, the external device 240 is in operative communication with the apparatus 200 (e.g., via the external component 142). In FIG. 4A, the external device 240 is also in operative communication with the at least one server computer 320 (e.g., via a network 310). The example on-line use configuration of FIG. 4A can be used, for example, by a recipient attempting to access a restricted service or functionality hosted by the at least one server computer 320. The example off-line use configuration of FIG. 4B can be used, for example, by a recipient attempting to access a restricted service or functionality hosted locally by the external device 240 (e.g., passwords; cryptocurrencies; personal information; use of the external device 240 as a “wallet”).

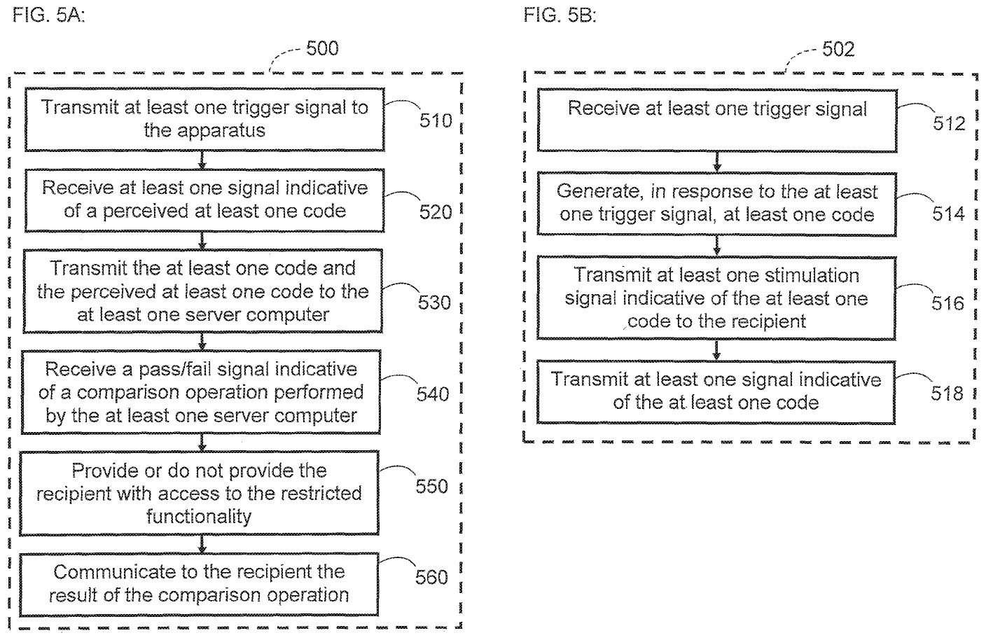

FIGS. 5A-5B are flow diagrams of example methods 500, 502 for authenticating a recipient's identity in the example on-line use configuration of FIG. 4A in accordance with certain embodiments described herein. The flow diagram of FIG. 5A refers to operations performed by the external device 240 in the example on-line use configuration of FIG. 4A and the flow diagram of FIG. 5B refers to operations performed by the apparatus 200 in the example on-line use configuration of FIG. 4A. Other example methods are compatible with other configurations in accordance with certain embodiments described herein. For example, another example method can refer to operations performed by the at least one server computer 320 in the example on-line use configuration of FIG. 4A. Other example methods are subsets of the operations of the method 500 performed by the external device 240, subsets of the operations of the method 502 performed by the apparatus 200, subsets of the operations performed by the at least one server computer 320, and/or combinations of at least some of the operations performed by the apparatus 200, the external device 240, and/or the at least one server computer 320.

In an operational block 510, the method 500 comprises transmitting at least one trigger signal 242 to the apparatus 200 (e.g., the implanted internal component 144 of a sensory prosthesis system) in response to a request from the recipient for access to a restricted functionality of the at least one server computer 320 (e.g., a restricted functionality accessible via the client application 410). In an operational block 512, the method 502 comprises receiving at least one trigger signal 242. For example, as schematically illustrated in FIG. 4A, the external device 240 transmits the at least one trigger signal 242 to the apparatus 200 (e.g., via the trust module 420 of the client application 410 running on the external device 240 and via the external component 142). In certain such embodiments in which the apparatus 200 comprises the implanted internal component 144 of the cochlear implant auditory prosthesis 100 and the external component 142 comprises the sound processing unit 126 of the cochlear implant auditory prosthesis 100, the sound processing unit 126 receives at least one trigger signal 242 from the trust module 420 and transmits the at least one trigger signal 242 to the implanted internal component 144 which receives the at least one trigger signal 242.

In certain embodiments, the method 500 and/or the method 502 further comprises presenting a query to the recipient, the query requesting entry of a perceived at least one code 434 from the recipient. For example, in the method 500, the trust module 420 can present the query to the recipient (e.g., using a display, speaker, or other output communication interface of the external device 240). For another example, in the method 502, the apparatus 200 can present the query to the recipient (e.g., via the at least one stimulation signal 252 provided to the recipient by the apparatus 200).

In an operational block 514, the method 502 further comprises, in response to the at least one trigger signal 242 and using the at least one secret 232, generating the at least one code 234. For example, the apparatus 200 can access the at least one secret 232 from the at least one storage device 230 and can generate the at least one code 234 using the at least one secret 232. In the operational block 516, the method 502 further comprises transmitting at least one stimulation signal 252 from the apparatus 200 to the recipient, the at least one stimulation signal 252 indicative of the at least one code 234. In the operational block 518, the method 502 further comprises transmitting at least one signal 432 indicative of the at least one code 234 from the apparatus 200 to the external device 240. For example, the apparatus 200 can transmit the at least one signal 432 via the external component 142 to the external device 240.

The recipient perceives the at least one stimulation signal 252 as a perceived at least one code 434 and the recipient communicates at least one signal 436 (e.g., at least one user input signal) to the trust module 420 of the client application 410 running on the external device 240 (e.g., via at least one input communication interface of the external device 240), the at least one signal 436 indicative of the perceived at least one code 434. In the operational block 520, the method 500 further comprises receiving the at least one signal 436 indicative of the perceived at least one code 434 from the recipient.

In an operational block 530, the method 500 further comprises transmitting the at least one code 234 and the perceived at least one code 434 to the at least one server computer 320. For example, as schematically illustrated by FIG. 4A, the trust module 420 can transmit the at least one signal 432 indicative of the at least one code 234 and at least one signal 438 indicative of the perceived at least one code 434 (e.g., via the network 310) to the at least one server computer 320. In certain embodiments, the at least one signal 436 and the at least one signal 438 are transmitted at substantially the same time (e.g., simultaneously), while in certain other embodiments, the at least one signal 436 and the at least one signal 438 are transmitted at substantially different times (e.g., sequentially to one another).

In certain such embodiments, as schematically illustrated by FIG. 4A, the at least one server computer 320 performs a comparison operation 440 which compares the at least one code 234 and the perceived at least one code 434. In an operational block 540, the method 500 further comprises receiving (e.g., by the trust module 420 of the external device 240) a pass/fail signal 442 from the at least one server computer 320, the pass/fail signal 442 indicative of a result of the comparison operation 440. If the comparison operation 440 determines that the at least one code 234 and the perceived at least one code 434 do not match one another, the authentication process fails (e.g., the recipient's identity is not authenticated) and the pass/fail signal 442 is indicative of the failure of the authentication process. If the comparison operation 440 determines that the at least one code 234 and the perceived at least one code 434 match one another, the authentication process succeeds (e.g., the recipient's identity is authenticated) and the pass/fail signal 442 is indicative of the success of the authentication process. In certain embodiments, if the at least one server computer 320 has information regarding the recipient's identity, the at least one server computer 320 can provide this information to the trust module 420 (e.g., with the pass/fail signal 442). In certain embodiments, if the at least one code 234 and the perceived at least one code 434 match one another, the at least one server computer 320 transmits an electronic token to the external device 240, the electronic token indicative of the successful authentication of the recipient's identity (e.g., to be used with other client applications; to be used in future transactions with the at least one server computer 320 providing the restricted functionality).

In an operational block 550, the method 500 further comprises, in response to the pass/fail signal 442, either providing the recipient with access to the restricted functionality (e.g., if the at least one code 234 and the perceived at least one code 434 match one another) or not providing the recipient with access to the restricted functionality (e.g., if the at least one code 234 and the perceived at least one code 434 do not match one another). In an operational block 560, the method 500 further comprises communicating to the recipient the result of the comparison operation 440. For example, the trust module 420 can set a pass/fail flag 444 to denote the success/failure of the authentication process and can cause a message 452 indicative of the success/failure of the authentication process to be communicated to the recipient (e.g., via a display, speaker, or other output communication interface of the external device 240).

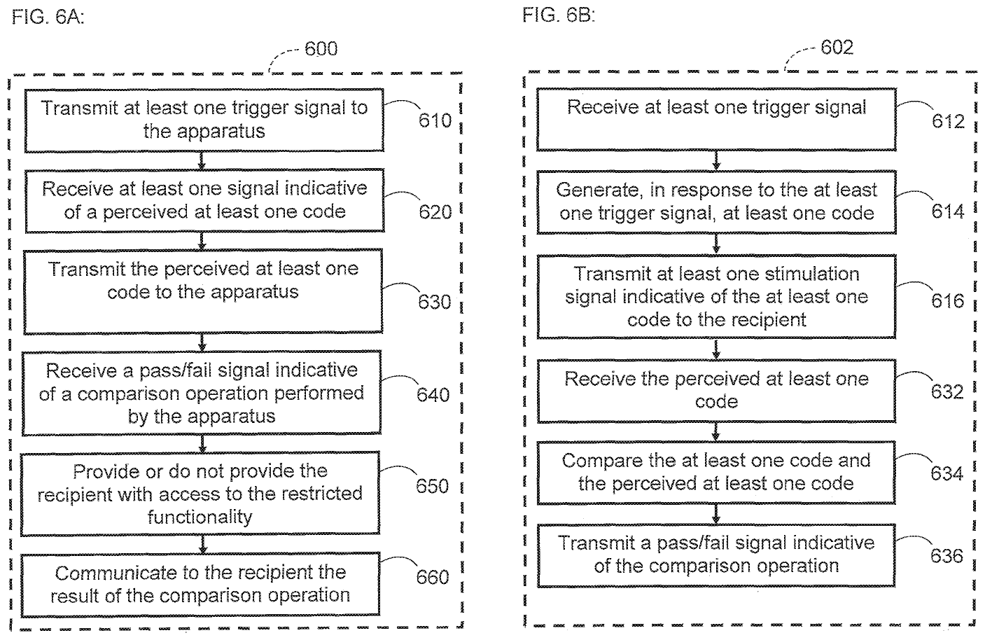

FIGS. 6A-6B are flow diagrams of example method 600, 602 for authenticating a recipient's identity in the example off-line use configuration of FIG. 4B in accordance with certain embodiments described herein. The flow diagram of FIG. 6A refers to operations performed by the external device 240 in the example off-line use configuration of FIG. 4B, and the flow diagram of FIG. 6B refers to operations performed by the apparatus 200 in the example off-line use configuration of FIG. 4B. Other example methods are compatible with other configurations in accordance with certain embodiments described herein. For example, other example methods are subsets of the operations of the method 600 performed by the external device 240, subsets of the operations of the method 602 performed by the apparatus 200, and/or combinations of at least some of the operations performed by the apparatus 200 and the external device 240.

In an operational block 610, the method 600 comprises transmitting at least one trigger signal 242 to the apparatus 200 (e.g., the implanted internal component 144 of a sensory prosthesis system) in response to a request from the recipient for access to a restricted functionality of the client application 410. In an operational block 612, the method 602 comprises receiving at least one trigger signal 242. For example, as schematically illustrated in FIG. 4B, the external device 240 transmits the at least one trigger signal 242 to the apparatus 200 (e.g., via the trust module 420 of the client application 410 running on the external device 240 and via the external component 142). In certain such embodiments in which the apparatus 200 comprises the implanted internal component 144 of the cochlear implant auditory prosthesis 100 and the external component 142 comprises the sound processing unit 126 of the cochlear implant auditory prosthesis 100, the sound processing unit 126 receives at least one trigger signal 242 from the trust module 420 and transmits the at least one trigger signal 242 to the implanted internal component 144 which receives the at least one trigger signal 242.

In certain embodiments, the method 600 and/or the method 602 further comprises presenting a query to the recipient, the query requesting entry of a perceived at least one code 434 from the recipient. For example, in the method 600, the trust module 420 can present the query to the recipient (e.g., using a display, speaker, or other output communication interface of the external device 240). For another example, in the method 602, the apparatus 200 can present the query to the recipient (e.g., via the at least one stimulation signal 252 provided to the recipient by the apparatus 200).

In an operational block 614, the method 602 further comprises, in response to the at least one trigger signal 242 and using the at least one secret 232, generating the at least one code 234. For example, the apparatus 200 can access the at least one secret 232 from the at least one storage device 230 and can generate the at least one code 234 using the at least one secret 232. In the operational block 616, the method 602 further comprises transmitting at least one stimulation signal 252 from the apparatus 200 to the recipient, the at least one stimulation signal 252 indicative of the at least one code 234.

The recipient perceives the at least one stimulation signal 252 as a perceived at least one code 434 and the recipient communicates at least one signal 436 (e.g., at least one user input signal) to the trust module 420 of the client application 410 running on the external device 240 (e.g., via at least one input communication interface of the external device 240), the at least one signal 436 indicative of the perceived at least one code 434. In the operational block 620, the method 600 further comprises receiving the at least one signal 436 indicative of the perceived at least one code 434 from the recipient.

In an operational block 630, the method 600 further comprises transmitting the perceived at least one code 434 to the apparatus 200. For example, as schematically illustrated by FIG. 4B, the trust module 420 can transmit at least one signal 438 indicative of the perceived at least one code 434 via the external component 142 to the apparatus 200 (e.g., the implanted internal component 144). In an operational block 632, the method 602 further comprises receiving the perceived at least one code 434 (e.g., from the external device 240).

In an operational block 634, the method 602 further comprises comparing the at least one code 234 and the perceived at least one code 434. For example, as schematically illustrated by FIG. 4B, the apparatus 200 can compare the at least one code 234 and the perceived at least one code 434 in a comparison operation 440. In an operational block 636, the method 602 further comprises transmitting a pass/fail signal 442 to the trust module 420 the pass/fail signal 442 indicative of a result of the comparison operation 440. In an operational block 640, the method 600 further comprises receiving the pass/fail signal. If the comparison operation 440 determines that the at least one code 234 and the perceived at least one code 434 do not match one another, the authentication process fails (e.g., the recipient's identity is not authenticated) and the pass/fail signal 442 is indicative of the failure of the authentication process. If the comparison operation 440 determines that the at least one code 234 and the perceived at least one code 434 match one another, the authentication process succeeds (e.g., the recipient's identity is authenticated) and the pass/fail signal 442 is indicative of the success of the authentication process.

In an operational block 650, the method 600 further comprises, in response to the pass/fail signal 442, either providing the recipient with access to the restricted functionality (e.g., if the at least one code 234 and the perceived at least one code 434 match one another) or not providing the recipient with access to the restricted functionality (e.g., if the at least one code 234 and the perceived at least one code 434 do not match one another). In an operational block 660, the method 600 further comprises communicating to the recipient the result of the comparison operation 440. For example, the trust module 420 can set a pass/fail flag 444 to denote the success/failure of the authentication process and can cause a message 452 indicative of the success/failure of the authentication process to be communicated to the recipient (e.g., via a display, speaker, or other output communication interface of the external device 240).

In certain embodiments, communications of the at least one code 234 and/or the at least one perceived code 434 between the apparatus 200, the at least one server computer 320, the external device 240, and/or the second external device 330 are transmitted across secure communication channels (e.g., trusted communication channels). For example, referring to FIGS. 4A-4B, the communications between the apparatus 200 and the trust module 420 of the external device 240 can be transmitted across secure communication channels. Referring to FIG. 4A, the communications between the apparatus 200 and the at least one server computer 320 and the communications between the trust module 420 and the at least one server computer 320 can also be transmitted across secure communication channels. In certain embodiments, the secure communication channels are achieved over an untrusted path using cryptographic techniques, examples of which include but are not limited to the Diffie-Helman (DH) key exchange, the Advanced Encryption Standard (AES) algorithm, or the Rivest-Shamir-Adleman (RSA) algorithm. For example, the apparatus 200 and the trust module 420 can execute key exchange prior to any of the communications described in reference to FIGS. 3A-3C and 4A-4B to generate a shared key or a key pair. The shared key or key pair can then be used for encryption/decryption of the communications described herein.

It is to be appreciated that the embodiments disclosed herein are not mutually exclusive and may be combined with one another in various arrangements.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree.

Certain embodiments described herein include methods which are performed by computer hardware, software or both, comprising one or more modules. The at least some of the hardware used for certain embodiments described herein can take a wide variety of forms, including processors, general-purpose computers, network servers, workstations, personal computers, mainframe computers and the like. The hardware running the software can include one or more input devices, such as a mouse, trackball, touchpad, and/or keyboard, a display, and computer-readable memory media, such as random-access memory (RAM) integrated circuits and a data storage device (e.g., tangible storage, non-transitory storage, flash memory, hard-disk drive). It will be appreciated that one or more portions, or all of the software code may be remote from the user and, for example, resident on a network resource, such as a LAN server, Internet server, network storage device, etc. The software code which configures the hardware to perform in accordance with certain embodiments described herein can be downloaded from a network server which is part of a local-area network or a wide-area network (such as the internet) or can be provided on a tangible (e.g., non-transitory) computer-readable medium, such as a CD-ROM or a flash drive. Various computer languages, architectures, and configurations can be used to practice the various embodiments described herein.

The invention described and claimed herein is not to be limited in scope by the specific example embodiments herein disclosed, since these embodiments are intended as illustrations, and not limitations, of several aspects of the invention. Any equivalent embodiments are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example embodiments disclosed herein, but should be defined only in accordance with the claims and their equivalents.

1. An apparatus comprising:
a housing configured to be implanted in or on a recipient; and
circuitry within the housing, the circuitry comprising at least one storage device configured to store at least one secret, the circuitry configured to:
generate, using the at least one secret, at least one code corresponding to the at least one secret; and
transmit at least one stimulation signal to the recipient, the at least one stimulation signal indicative of the at least one code.

2. The apparatus of claim 1, wherein the apparatus comprises an implantable portion of a medical implant system.

3. The apparatus of claim 1, wherein the apparatus comprises an implantable portion of a sensory prosthesis system.

4. The apparatus of claim 3, wherein the sensory prosthesis comprises a cochlear implant system.

5. The apparatus of claim 3, further comprising an external portion of the sensory prosthesis system, the external portion external to the recipient and in wireless communication with the implantable portion, the external portion configured to receive the at least one trigger signal from an external device and to provide the at least one trigger signal to the implantable portion.

6. The apparatus of claim 1, wherein the at least one secret comprises information indicative of the identity of the apparatus.

7. The apparatus of claim 6, wherein the circuitry is further configured to generate the at least one code by inputting the at least one secret into at least one algorithm configured to output the at least one code.

8. The apparatus of claim 1 wherein the at least one secret comprises a private key and the at least one code comprises a public key.

9. The apparatus of claim 1, wherein the circuitry is further configured to wirelessly receive at least one trigger signal and, in response to the received at least one trigger signal, to generate the at least one code and transmit the at least one stimulation signal.

10. An apparatus comprising:
at least one first communication interface configured to wirelessly communicate with a system comprising at least one implant in or on a recipient, the implant configured to:
generate, using at least one secret, at least one code corresponding to the at least one secret; and
transmit at least one stimulation signal to the recipient, the at least one stimulation signal indicative of the at least one code; and
at least one second communication interface configured to receive at least one user input signal from the recipient, the at least one user input signal indicative of the at least one code,
the at least one first communication interface further configured to transmit at least one trigger signal to the at least one implant, the at least one trigger signal configured to initiate said generating the at least one code by the at least one implant.

11. The apparatus of claim 10, wherein the at least one first communication interface is further configured to transmit the at least one code from the received at least one user input signal to the at least one implant.

12. The apparatus of claim 10, further comprising at least one third communication interface configured to communicate with at least one server computer over a secure communication link, wherein the at least one third communication interface is further configured to transmit the at least one code from the received at least one user input signal to the at least one server computer.

13. A method comprising:
accessing at least one secret stored on a device implanted in or on a recipient;
generating at least one first code corresponding to the at least one secret; and
transmitting at least one stimulation signal to the recipient, the at least one stimulation signal indicative of the at least one first code.

14. The method of claim 13, wherein generating the at least one first code and transmitting the at least one stimulation signal to the recipient are performed by the device implanted in or on the recipient.

15. The method of claim 13, wherein generating the at least one first code comprises applying at least one algorithm to the at least one secret.

16. The method of claim 13, further comprising initiating said generating the at least one first code in response to at least one trigger signal from a mobile device.

17. The method of claim 13, further comprising using the device to:
receive at least one second code from the mobile device; and
determine whether the at least one second code corresponds to the at least one first code.

18. The method of claim 13, further comprising transmitting at least one signal to the recipient, the at least one signal indicative of whether the at least one second code corresponds to the at least one first code.

19. A method comprising:
transmitting at least one trigger signal to a device implanted in or on a recipient, the device configured to respond to the at least one trigger signal by:
using at least one secret stored on the device to generate at least one code; and
transmitting at least one stimulation signal to the recipient, the at least one stimulation signal indicative of the at least one code;
receiving at least one first signal indicative of a perceived at least one code perceived by the recipient in response to the at least one stimulation signal;
transmitting at least one second signal indicative of the perceived at least one code;
receiving at least one comparison signal indicative of whether the at least one code and the perceived at least one code match one another or not; and
either providing the recipient with access to a restricted functionality in response to the at least one comparison signal being indicative of the at least one code matching the perceived at least one code or not providing the recipient with the access in response to the at least one comparison signal being indicative of the at least one code not matching the perceived at least one code.

20. The method of claim 19, wherein transmitting the at least one second signal comprises transmitting the at least one second signal to the device, and receiving the at least one comparison signal comprises receiving the at least one comparison signal from the device.

21. The method of claim 19, wherein transmitting the at least one second signal comprises transmitting the at least one second signal to at least one server computer, and receiving the at least one comparison signal comprises receiving the at least one comparison signal from the at least one server computer.

22. The method of claim 19, further comprising communicating to the recipient at least one third signal indicative of the at least one comparison signal.
